(19) United States
(12) Patent Application Publication
(10) Pub. No.: US 2026/0014015 A1
(43) Pub. Date: Jan. 15, 2026

(54) THERAPEUTIC SHOULDER BRACE

(71) Applicant: Shin-ichi NAKAMITSU, Fukuoka (JP)

(72) Inventor: Shin-ichi NAKAMITSU, Fukuoka (JP)

(21) Appl. No.: 19/078,723

(22) Filed: Mar. 13, 2025

(30) Foreign Application Priority Data
Jul. 10, 2024 (JP) 2024-110821

(51) Int. Cl. A61F 5/37

(52) U.S. Cl. CPC A61F 5/3738; A61F 5/373

(57) ABSTRACT

To provide a therapeutic shoulder brace having a strap applied to a shoulder, which has a simple configuration and can reliably prevent the therapeutic shoulder brace from slipping off the shoulder. A therapeutic shoulder brace including a shoulder portion placed on an affected shoulder and a strap hung so as to hold an upper limb in a physiological basic position, wherein in a developed plan view of the therapeutic shoulder brace developed in the length direction with the shoulder portion at the center, the strap has a bent region that includes the shoulder portion and extends inwardly in a convex shape, and a straight region that is connected to a front end and a back end of the bent region and extends linearly in the front-back direction is provided.

### FIELD

The present invention relates to a therapeutic shoulder brace applied on a patient suffering from a shoulder injury such as fracture, dislocation, or ligament injury.

### BACKGROUND

Conventionally, as therapeutic shoulder braces, static braces have been generally employed to hold and fix shoulders and arms for a long period of time after injuries such as proximal humerus fracture, rotator cuff tear, acromioclavicular joint dislocation, shoulder joint dislocation, and distal clavicle fracture. In such shoulder braces, there has been a problem that contracture of bones and muscles occurs due to long-term fixation that impedes rehabilitation, and as a result, dysfunction is likely to occur.

The above problems have been solved by therapeutic shoulder braces described in International Publications No. WO2015-166561 and No. WO2017-017815. The therapeutic shoulder braces described in International Publications No. WO2015-166561 and No. WO2017-017815 have a ring-shaped strap (also referred to as “sling”) applied on an affected shoulder, and in an applied state, the strap is hung so as to descend from a distal clavicle to an elbow proximal part of a forearm on an ulnar side, passes through a lower surface side of the forearm from the ulnar side to a radial side, traverses the front side of an upper arm from the radial side of the forearm, and then returns to the distal clavicle through an axilla. By hanging in this manner, an upper limb can be held at a physiological fundamental position. The upper limb held in the physiological fundamental position can be moved in a front-back direction like a single pendulum movement after using the static brace only at an initial stage after injury or by applying this strap from immediately after injury. Accordingly, rehabilitation can be performed while applying the therapeutic shoulder brace. The straps described in International Publications No. WO2015-166561 and No. WO2017-017815 have both a fixing function and an early rehabilitation function.

On the other hand, the straps described in International Publications No. WO2015-166561 and No. WO2017-017815 are placed at the distal clavicle, and thus there is a problem that the straps easily slip off the shoulder. In particular, unlike static braces, the braces are used to promote movement of the upper limb, so that the straps more easily slip off the shoulder with the movement. Therefore, International Publication No. WO2015-166561 discloses that an anti-slip shoulder pad, an adhesive sheet, or the like is provided on a back surface of a shoulder portion of the strap. International Publication No. WO2017-017815 discloses a countermeasure such that a layer member in which shoulder pads placed on both shoulders are connected is provided and the strap are fixed to the layer member.

However, the shoulder pads or the like described in International Publication No. WO2015-166561 cannot reliably prevent the straps from slipping off, and are not particularly effective for patients with sloped shoulders. In addition, although the layer member applied on both shoulders described in International Publication No. WO2017-017815 can reliably prevent the strap from slipping off, the applying method thereof is complicated, and the shoulder braces become large as a whole and have a complicated structure.

In view of the above problems, an object of the present invention is to provide a therapeutic shoulder brace having a strap applied on an affected shoulder to hold an upper limb in a physiological fundamental position and to reliably prevent slipping off of the strap from a shoulder with a simple configuration.

### SUMMARY

In order to solve the above problems, the present invention provides the following configurations. Numbers in brackets are reference signs indicated in the drawings described later and are denoted for reference.
An aspect [1] of the present invention is a therapeutic shoulder brace (1, 1A) comprising a shoulder portion (11a) placed on an affected shoulder and a strap (10, 10A) hung so as to hold an upper limb in a physiological basic position, wherein in a developed plan view of the therapeutic shoulder brace (1, 1A) developed in a length direction with the shoulder portion (11a) at the center, the strap (10) has a bent region (11) that includes the shoulder portion (11a) and extends inwardly in a convex shape, and a straight region (12) that is connected to a front end (11d) and a back end (11e) of the bent region (11) and extends linearly in a front-back direction.
[2] In the aspect [1], it is preferable that the therapeutic shoulder brace includes an underarm band (20) connecting a vicinity of a front end (11b) and a vicinity of a back end (11c) of the shoulder portion (11a) of the strap (10, 10A) through an axilla.
[3] In the aspect [1], it is preferable that when the shoulder portion (11a) is placed on a shoulder, the shoulder portion (11a) is inclined in a same direction as an inclination of the shoulder.
[4] In any of the aspects [1] to [3], it is preferable that the strap (10) is ring-shaped, and in an applied state, the strap (10) descends from the front end (11b) of the shoulder portion (11a) to an elbow proximal part of a forearm on an ulnar side, passes through a lower surface side of the forearm from the ulnar side to a radial side, traverses a front side of an upper arm from the radial side of the forearm, and connects to a back end (11c) of the shoulder portion (11a) through the axilla.
[5] In any of the aspects [1] to [3], it is preferable that the therapeutic shoulder brace includes an elbow pad (30) for supporting a forearm, wherein the strap (10A) has a band shape including a first end (13) and a second end (14), and in an applied state, the strap (10A) descends from the front end (11b) of the shoulder portion (11a) to an elbow proximal part of the forearm on an ulnar side, the first end (13) of the strap (10A) is fixed to the elbow pad (30), and the second end (14) of the strap (10A) is fixed to the elbow pad (30) from the back end (11c) of the shoulder portion (11a) through an axilla, traverses a front side of an upper arm, and connects to the radial side of the forearm.
[6] In the aspect [5], it is preferable that the second end (14) of the strap (10A) is fixed to the elbow pad (30) at a position closer to a front side than a longitudinal midpoint (M) of the elbow pad (30).

### Effect of the Invention

According to the present invention, in a therapeutic shoulder brace having a strap applied on an affected shoulder to hold an upper limb in a physiological fundamental position, it is possible to reliably prevent slipping off of the strap from a shoulder with a simple configuration.

### BRIEF DESCRIPTION OF DRAWINGS

FIG. 1 is a schematic perspective view illustrating a state in which a therapeutic shoulder brace according to a first embodiment of the present invention is applied on a patient;

FIG. 2A is a schematic perspective view illustrating only the therapeutic shoulder brace extracted from FIG. 1;

FIG. 2B is a schematic perspective view of the therapeutic shoulder brace hung in a different way from that of FIG. 2A;

FIG. 3 is a schematic perspective view similar to that of FIG. 1, illustrating a therapeutic shoulder brace according to a second embodiment of the present invention;

FIG. 4A is a schematic perspective view illustrating an example of the shape of the therapeutic shoulder brace in FIG. 1 in an unapplied state;

FIG. 4B is a schematic front view corresponding to FIG. 4A;

FIG. 4C is a schematic front view illustrating differences from conventional straps;

FIGS. 5A to 5F are developed plan views cut along a chain line in FIG. 4A and developed in a longitudinal direction and are examples of the therapeutic shoulder brace;

FIGS. 6A to 6C illustrate another embodiment of the therapeutic shoulder brace according to the present invention and are schematic perspective views similar to that of FIG. 4A;

FIG. 7A is a top view for explaining a state in which the therapeutic shoulder brace according to the present invention is used; and

FIG. 7B is a side view corresponding to FIG. 7A.

### DESCRIPTION OF EMBODIMENTS

Embodiments of the present invention will be described below with refence to exemplary drawings. It is to be noted that identical or similar constituent elements in the respective embodiments are denoted by like or similar reference signs.

In the present specification, “front” and “back” when referring to as “forward”, “front end”, “back end”, and “backward” are the same as the front-back direction of a body.

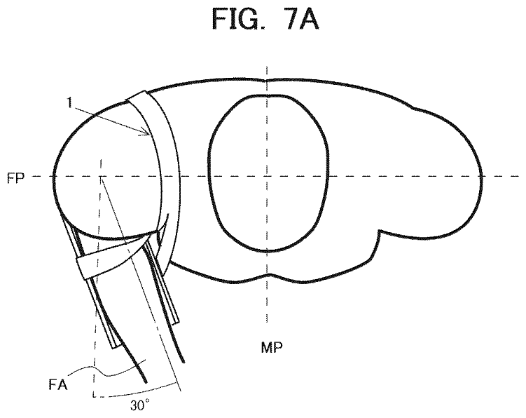

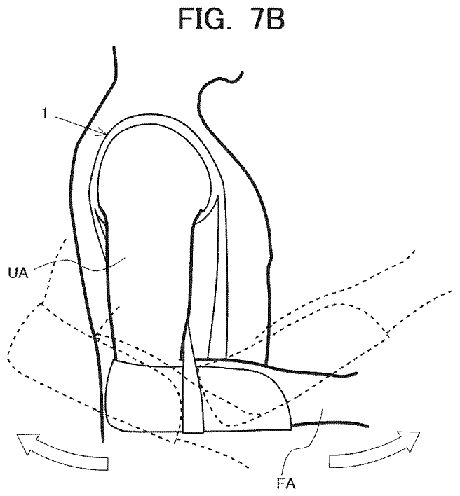

In addition, in the left-right direction of the body, the median plane side as viewed from shoulders is referred to as “inside” and “inward”, and the opposite side to the median plane is referred to as “outside” and “outward”. The median plane is denoted by a reference sign MP in FIG. 7A.

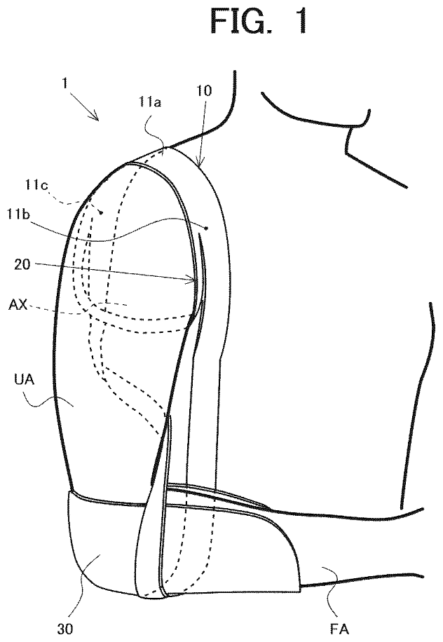

FIG. 1 is a schematic perspective view illustrating a state in which a therapeutic shoulder brace 1 according to a first embodiment of the present invention is applied on a patient. In FIG. 1, the right shoulder is the affected shoulder.

A preferred example of the therapeutic shoulder brace 1 according to the first embodiment includes a ring-shaped strap 10, an underarm band 20, and an elbow pad 30. The strap 10 and the underarm band 20 are formed of a flexible material having appropriate deformability and strength. The elbow pad 30 is provided to stably support a forearm FA from below, and is formed of a material having a degree of hardness capable of stably supporting the forearm FA.

The strap 10 illustrated in FIG. 1 is a closed ring member having a suitable length, width, and thickness. A shoulder portion 11a located at the uppermost part in an applied state of the strap 10 is placed on the affected shoulder (including the vicinity of the front and back of the shoulder). The width of the strap 10 is substantially constant in the length direction, but does not need to be strictly constant. In FIG. 1, the underarm band 20 is branched from the shoulder portion 11a of the strap 10, so that the shoulder portion 11a is wider than other portions by the width of the underarm band 20; however, this is only an example. Although not illustrated in FIG. 1, it is preferable to provide the strap 10 with an adjustment tool capable of adjusting its length according to the physical constitution of a patient.

The therapeutic shoulder brace 1 is applied so that a patient keeps the physiological fundamental position in which the elbow joint is bent forward by 90°. The physiological fundamental position is a position in which the upper limb is naturally lowered along the body, the upper limb is hung with the elbow positioned substantially at a right angle, the flexion and extension of the shoulder are 0°, the abduction and adduction are 0°, and the internal rotation is 30° (see FIG. 7A). Since the axis of the scapula is directed outward from a frontal plane FP by about 30°, the forearm FA is oriented in a direction that is internally rotated by 30° with respect to a direction perpendicular to the frontal plane FP (for convenience of description, this direction of internal rotation is also referred to as “forward”). Note that these angles are slightly different depending on the physical constitution of an individual and the movement of muscles, and thus these angles are not exact numerical values and may have a range of a certain degree (for example, about plus or minus 10%). The physiological fundamental position is achieved by the hanging mode of the strap 10.

The hanging mode of the strap 10 for holding the upper limb in a physiological fundamental position is disclosed in International Publications No. WO2015-166561 and No. WO2017-017815, and is as follows. The strap 10 descends substantially along a sagittal plane (a plane parallel to a median plane) from a front end 11b of the shoulder portion 11a to an elbow proximal part of the forearm FA on an ulnar side, passes through a lower surface side of the forearm FA from the ulnar side to a radial side, traverses the front side of the upper arm UA from the radial side of the forearm FA, and is then connected to a back end 11c of the shoulder portion 11a through an axilla. When the forearm FA is supported by the elbow pad 30, the strap 10 is applied to pass through the outside of the elbow pad 30.

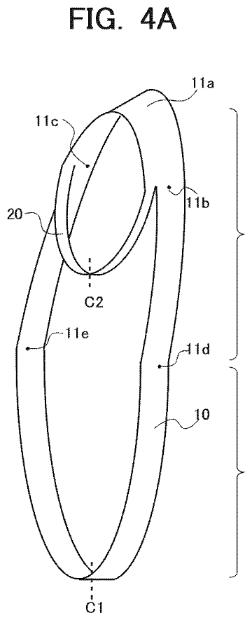

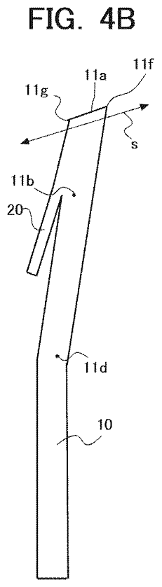

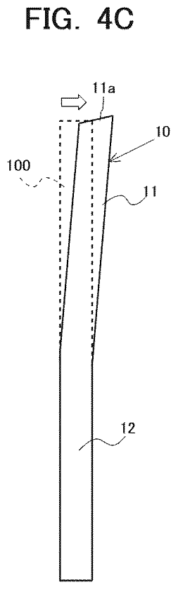

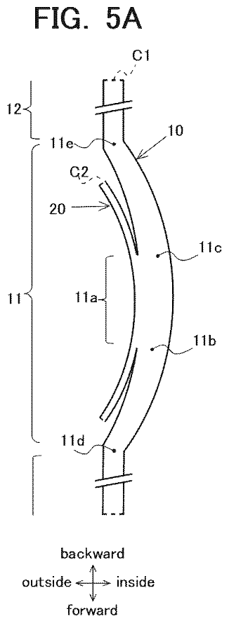

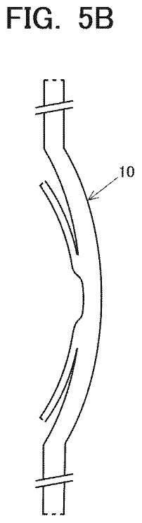

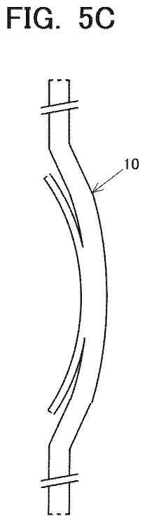

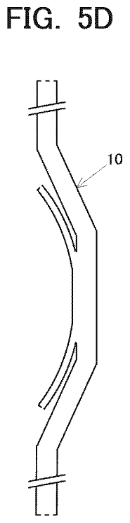

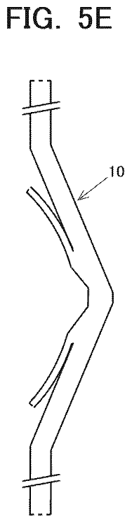

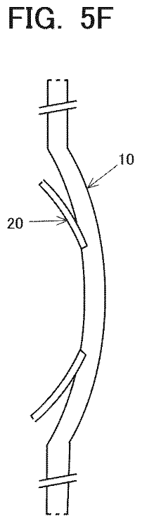

Although it is not clear in FIG. 1, in the strap 10, the upper portion (approximately the upper half) including the shoulder portion 11a is bent inward with respect to the lower portion, that is, inclined inward with respect to the sagittal plane. This structure prevents the strap 10 from slipping off by placing the shoulder portion 11a more on the inner side than the distal clavicle. This structure will be described in detail with reference to FIG. 4 and FIG. 5.

The underarm band 20 connects the front end 11b and the back end 11c of the shoulder portion 11a of the strap 10 through an axilla AX. In the example of FIG. 1, the underarm band 20 is formed by branching from a portion of the strap 10. The underarm band 20 also serves to prevent the strap 10 from slipping off the shoulder. Since the underarm band 20 passes through the axilla AX, it is preferable to have a width that does not interfere with the movement of the upper limb during rehabilitation.

In the illustrated example, the elbow pad 30 has a shape that covers the lower surface and the side surface of the periphery of the elbow and an elbow proximal part of the forearm, and is open upward and forward. For example, the length of the elbow pad 30 is half or slightly longer than half of the forearm FA. In the illustrated example, the height of the side surface of the elbow pad 30 is substantially the same as the thickness of the elbow proximal part of the forearm FA, but may be any height as long as the forearm FA can be stably supported.

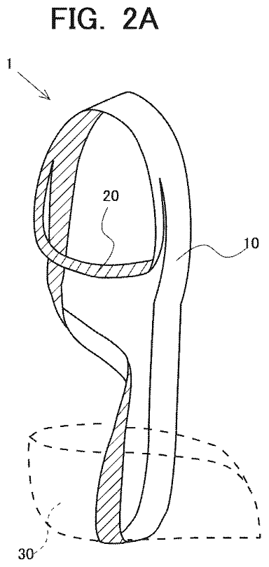

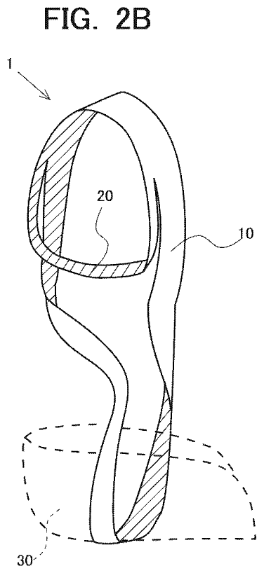

FIG. 2A is a schematic perspective view of the therapeutic shoulder brace 1 extracted from FIG. 1 in a state of being applied. While FIG. 2B is the same view as FIG. 2A, it is slightly different in the way the straps 10 are hung. The elbow pad 30 is represented in a chain line for better understanding of the state of the strap 10. Further, the back side of the strap 10 is shown by hatching.

As illustrated in FIG. 2A and FIG. 2B, the strap 10 in an applied state is not in a simple ring shape, but a constriction is formed at the center of the ring and the strap is twisted as a whole around this constriction. This twisting works to control the upper arm UA and the forearm FA to be held at the physiological fundamental position described above.

The strap 10 in an applied state is twisted or the front and back sides are reversed at several positions (including front-back reversal like a Mobius strip). While FIG. 2A and FIG. 2B illustrate examples in which the paths through which the straps 10 pass are the same, the front and back sides are partially different. In FIG. 2A, the back side of the strap 10 is exposed on the lower side of the elbow pad 30; however, the front side of the strap 10 is exposed in FIG. 2B. From the viewpoint of therapeutic effect, there is no problem in employing either of the configurations in FIG. 2A and FIG. 2B.

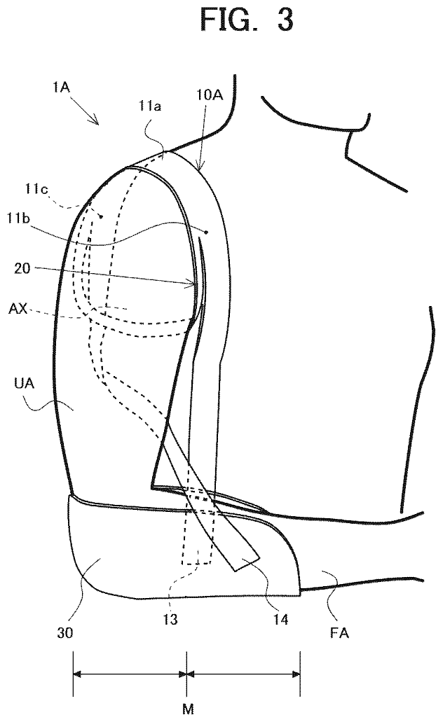

FIG. 3 is a schematic perspective view similar to that of FIG. 1, illustrating a therapeutic shoulder brace 1A according to a second embodiment of the present invention. The second embodiment differs from the first embodiment in the configuration of a strap 10A. Since the underarm band 20 and the elbow pad 30 other than the strap 10 are the same as those of the first embodiment, descriptions thereof will be omitted.

While the strap 10A according to the second embodiment is not a closed ring member, it is band-shaped with a first end 13 and a second end 14. On the front side of the shoulder portion 11a of the strap 10A, the strap 10A descends substantially along a sagittal plane from the front end 11b of the shoulder portion 11a to the elbow proximal part of the forearm FA on the ulnar side, and the first end 13 of the strap 10A is fixed to the ulnar outer surface of the elbow pad 30 by suitable means.

On the other hand, on the back side of the shoulder portion 11a of the strap 10A, the strap 10A passes from the back end 11c of the shoulder portion 11a through the axilla, traverses the front of the upper arm UA, and is connected to the radial side of the forearm FA, and the second end 14 of the strap 10A is fixed to the radial outer surface of the elbow pad 30 by suitable means. The second end 14 of the strap 10A is preferably fixed to the elbow pad 30 at a position closer to the front side than a longitudinal midpoint M of the elbow pad 30. By fixing the second end 14 at such a position, a more stable forearm support is possible.

The means for fixing the first end 13 and the second end 14 of the strap 10A to the elbow pad 30 may be non-detachable or detachable. Examples of non-detachable means include adhesion, sewing, welding, and the like. For example, there is a hook-and-loop fastener as detachable means. When the detachable means is used, it is also possible to adjust the fixing position. In addition, in order to more firmly fix the first end 13 and the second end 14 to each other, each of the first end 13 and the second end 14 may be further extended from the illustrated position and fixed so as to be wound around the elbow pad 30.

The function of the strap 10A according to the second embodiment is the same as that of the strap 10 according to the first embodiment described above, and the upper limb can be held in a physiological fundamental position.

In the following descriptions, while the strap 10 according to the first embodiment will be described mainly as an example, it can also be applied to the strap 10A according to the second embodiment.

FIG. 4A is a schematic perspective view illustrating an example of the shape of the therapeutic shoulder brace 1 of FIG. 2B in an unapplied state, and FIG. 4B is a schematic front view of FIG. 4A. FIG. 4C is a schematic front view illustrating differences from conventional straps.

In FIG. 4A to FIG. 4C, the strap 10 of the therapeutic shoulder brace 1 is illustrated in a vertical substantially elliptical shape with the shoulder portion 11a positioned at the uppermost part.

As illustrated in FIG. 4A, the strap 10 is not a uniform ring over the entire circumference, but has an upper bent region 11 and a lower straight region 12. A front end of the straight region 12 is connected to a front end 11d of the bent region 11, and a back end of the straight region 12 is connected to a back end 11e of the bent region 11. Preferably, the bent region 11 and the straight region 12 are connected with the same width. The boundary between the upper bent region 11 and the lower straight region 12 does not need to be a position that strictly bisects the length of the strap 10 and may be biased upward or downward, for example.

In the case of the band-shaped strap 10A illustrated in FIG. 3, it can be regarded as substantially the same as that obtained by cutting the straight region 12 in the strap 10 of FIG. 4A at an appropriate position.

As illustrated in FIG. 4B, in a front view, the lower straight region 12 extends in the vertical direction, and the upper bent region 11 is shifted inward (toward the median plane side) with respect to the vertical direction from the lower side toward the upper side. As a result, the shoulder portion 11a of the strap 10 is located several centimeters inward from the distal clavicle, so that the strap 10 is less likely to slip off.

Furthermore, in a front view, it is preferable that an inner edge 11f of the shoulder portion 11a is higher than an outer edge 11g. This results in the shoulder portion 11a being inclined in the same direction as an inclination s of the shoulder when the shoulder portion 11a is placed on the shoulder. As a result, the strap 10 can be brought into close contact with the shoulder over the entire width of the shoulder portion 11a, thereby contributing to prevention of slipping off of the strap 10.

A solid line in FIG. 4C represents a front view of only the strap 10 excluding the underarm band 20 in FIG. 4B. A dotted line 100 represents a strap shape in which the straight region 12 extends straight toward the upper part. The strap 10 according to the present invention has the upper part as the bending region 11, so that the strap 10 is inclined inward in a front view, and the uppermost part (the shoulder portion 11a) is shifted inward by an amount indicated by an open arrow. This displacement is about 2 to 4 centimeters, for example. It can also be said that a spacious region is formed outside the shoulder portion 11a by the shoulder portion 11a of the strap 10 shifting inward, and the underarm band 20 is placed in this spacious region.

FIG. 5A is a developed plan view in which the therapeutic shoulder brace 1 is developed in a longitudinal direction with the shoulder portion 11a at the center by cutting the strap 10 and the underarm band 20, respectively, at the portions represented by chain lines C1 and C2 in FIG. 4A. FIG. 5B to FIG. 5F are developed plan views similar to FIG. 5A, respectively illustrating modified modes of the therapeutic shoulder brace 1. In FIG. 5A, the front-back direction and the inward-outward direction in an applied state are represented by arrows.

In the developed plan view of FIG. 5A, the strap 10 has the bent region 11 including the shoulder portion 11a, and the straight region 12 connected to the front end 11d and the back end 11e of the bent region 11 and extends linearly in the front-back direction. The bent region 11 includes the shoulder portion 11a substantially at the center in the front-back direction. In the developed plan view, the bent region 11 extends inwardly in a convex shape. The strap 10 of FIG. 5A and FIG. 5B has a shape in which the inner and outer edges of the bent region 11 are curved like an arc or elliptical arc as a whole. In FIG. 5A, the bent region 11 extends to gradually become wider from both ends toward the shoulder portion 11a. In FIG. 5B, the bent region 11 extends from both ends to the shoulder portion 11a with substantially the same width, and is also substantially the same width in the shoulder portion 11a except for the branch portions of the underarm band 20.

The strap 10 of FIG. 5C linearly extends obliquely inward by a predetermined length from both ends of the bent region 11 toward the shoulder portion 11a, and then transitions to a curved shape.

The strap 10 of FIG. 5D linearly extends obliquely inward by a predetermined length from both ends of the bent region 11 toward the shoulder portion 11a, and the shoulder portion 11 linearly extends in the front-back direction.

The strap 10 of FIG. 5E linearly extends inward from both ends of the bent region 11 to the center of the shoulder portion 11a, and the entire strap 10 has a V-shape as a whole.

In FIG. 5F, the shape of the strap 10 is substantially the same as that of FIG. 5B; however, the underarm band 20 is formed of a member separate from that of the strap 10, and both ends are fixed to the strap 10.

In the developed plan views of FIG. 5A to FIG. 5F, the straight region 12 extends to be located on a straight line in the front-back direction.

By providing the bent region 11 in the strap 10, the area where the strap 10 is in contact with the body at the shoulder and in the front and back thereof is increased as compared with the case where the strap 10 includes only the straight region without the bent region. As a result, the friction force between the strap 10 and the body increases, making it difficult to slip off. The shape of the strap 10 may have various modified modes other than those illustrated in FIG. 5A to FIG. 5F.

In the developed plan view, the underarm band 20 is drawn in a simplified manner and is not intended to have a specific shape. The branch position of the underarm band 20 from the strap 10 (or the attachment position to the strap 10), width, length, thickness, and the like are designed so as to obtain an appropriate shape that runs smoothly in the axilla.

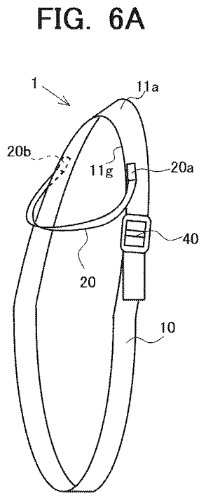

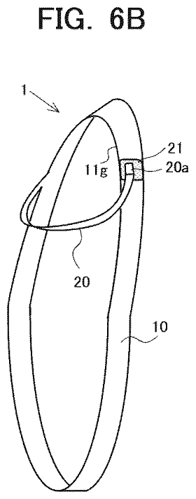

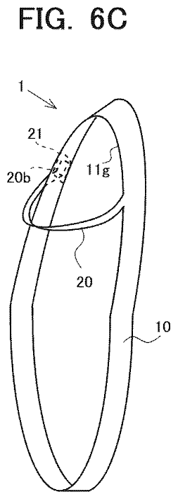

FIG. 6A, FIG. 6B, and FIG. 6C are schematic perspective views illustrating still another embodiment of the therapeutic shoulder brace 1 according to the present invention in an unapplied state similar to FIG. 4A. The therapeutic shoulder brace 1 of FIG. 6A has a fastener 40 for making the strap 10 ring-shaped. The fastener 40 also serves as an adjuster for the length of the strap 10. The fastener 40 is also applicable to the embodiment of FIG. 1 or FIG. 3 described above.

Further, in the embodiment of FIG. 6A, the underarm band 20 is formed of a separate member from that of the strap 10, and one end 20a thereof is fixed to the vicinity of the front end of the shoulder portion 11a and the other end 20b thereof is fixed to the vicinity of the back end of the shoulder portion 11a by suitable means. The fixing positions of both ends 20a and 20b of the underarm band 20 are preferably positions close to the outer edge 11g of the strap 10, but are not limited thereto. Other configurations are substantially the same as those of the embodiment illustrated in FIG. 1.

In the therapeutic shoulder brace 1 of FIG. 6B, the underarm band 20 branches from the strap 10 at the back end thereof, and the front end 20a is detachably attached to the strap 10. For example, the attachment position of the underarm band 20 can be adjusted by providing one member of a hook-and-loop fastener 21 on the front end 20a and providing the other member of the hook-and-loop fastener 21 on the strap 10. Thereby, the length of the underarm band 20 can be adjusted. Instead of a hook-and-loop fastener, a magnet or a button may be used.

FIG. 6C is a modified mode of FIG. 6B in which the underarm band 20 branches from the strap 10 at the front end thereof and a back end 20b is detachably attached to the strap 10.

FIG. 7A is a top view for explaining a state in which the therapeutic shoulder brace 1 according to the present invention is used, and FIG. 7B is a side view thereof. As illustrated in FIG. 7A, by appropriately applying the therapeutic shoulder brace 1 according to the present invention, a force for controlling the positions of the forearm FA and the upper arm of a patient is exerted. As a result, the internal rotation of 30° of the physiological fundamental position can be maintained with the elbow joint bent at a right angle. Various movements of the trunk in this state (for example, twisting of the waist during walking, forward bending of the trunk during standing, and the like) can trigger a passive movement in the front-back direction by forward flexion and backward extension as illustrated in FIG. 7B. Since the movement in the lateral direction (adduction/abduction direction) is suppressed by using the therapeutic shoulder brace 1, the passive movement becomes a single pendulum movement.

This single pendulum movement is limited in a safe direction. In this case, there is no movement in a prohibited direction that may cause dislocation of fractures or damage on muscles. Thus, by using a static brace only at an early stage after injury or from immediately after injury and then applying the therapeutic shoulder brace according to the present invention to perform a single pendulum movement, preliminary rehabilitation can be started. Since the patient's upper limb is not completely fixed over a long period of time, the risk of contracture can be eliminated.

The slip-off prevention structure of the therapeutic shoulder brace according to the present invention is a relatively simple structure in which the shoulder portion of the strap is displaced inward, but is very effective in preventing slipping off a shoulder. In addition, although the shoulder portion of the strap moves in the front-back direction with respect to a shoulder in accordance with the above-described single pendulum movement (see FIG. 7B), there is an advantage that the strap according to the present invention smoothly moves in the front-back direction as compared with a case where a conventional shoulder pad or a member applied on both shoulders is added to the strap. Furthermore, the strap according to the present invention is easy to apply. As a result, a patient can perform rehabilitation without worrying about the strap slipping off. This greatly reduces the stress on the patient.

Although the therapeutic shoulder brace according to the present invention has been described by presenting several embodiments, embodiments carried out by combining characteristic configurations of the respective embodiments can be also made and such embodiments are also included in the scope of the present invention. Furthermore, other various modifications can be made so long as complying with the spirit of the prevent invention.

What is claimed is:

1. A therapeutic shoulder brace comprising a shoulder portion placed on an affected shoulder and a strap hung so as to hold an upper limb in a physiological basic position, wherein
in a developed plan view of the therapeutic shoulder brace developed in a length direction with the shoulder portion at a center, the strap has a bent region that includes the shoulder portion and extends inwardly in a convex shape, and a straight region that is connected to a front end and a back end of the bent region and extends linearly in a front-back direction.

2. The therapeutic shoulder brace according to claim 1, comprising an underarm band connecting a vicinity of a front end and a vicinity of a back end of the shoulder portion of the strap through an axilla.

3. The therapeutic shoulder brace according to claim 1, wherein when the shoulder portion is placed on a shoulder, the shoulder portion is inclined in a same direction as an inclination of the shoulder.

4. The therapeutic shoulder brace according to claim 1, wherein the strap is ring-shaped, and in an applied state, the strap descends from a front end of the shoulder portion to an elbow proximal part of a forearm on an ulnar side, passes through a lower surface side of the forearm from the ulnar side to a radial side, traverses a front side of an upper arm from the radial side of the forearm, and connects to a back end of the shoulder portion through an axilla.

5. The therapeutic shoulder brace according to claim 2, wherein the strap is ring-shaped, and in an applied state, the strap descends from a front end of the shoulder portion to an elbow proximal part of a forearm on an ulnar side, passes through a lower surface side of the forearm from the ulnar side to a radial side, traverses a front side of an upper arm from the radial side of the forearm, and connects to a back end of the shoulder portion through an axilla.

6. The therapeutic shoulder brace according to claim 3, wherein the strap is ring-shaped, and in an applied state, the strap descends from a front end of the shoulder portion to an elbow proximal part of a forearm on an ulnar side, passes through a lower surface side of the forearm from the ulnar side to a radial side, traverses a front side of an upper arm from the radial side of the forearm, and connects to a back end of the shoulder portion through an axilla.

7. The therapeutic shoulder brace according to claim 1, comprising an elbow pad for supporting a forearm, wherein the strap has a band shape including a first end and a second end, and in an applied state, the strap descends from a front end of the shoulder portion to an elbow proximal part of the forearm on an ulnar side, the first end of the strap is fixed to the elbow pad, and the second end of the strap is fixed to the elbow pad from a back end of the shoulder portion through an axilla, traverses a front side of an upper arm, and connects to a radial side of the forearm.

8. The therapeutic shoulder brace according to claim 2, comprising an elbow pad for supporting a forearm, wherein the strap has a band shape including a first end and a second end, and in an applied state, the strap descends from the front end of the shoulder portion to an elbow proximal part of the forearm on an ulnar side, the first end of the strap is fixed to the elbow pad, and the second end of the strap is fixed to the elbow pad from the back end of the shoulder portion through an axilla, traverses a front side of an upper arm, and connects to a radial side of the forearm.

9. The therapeutic shoulder brace according to claim 3, comprising an elbow pad for supporting a forearm, wherein the strap has a band shape including a first end and a second end, and in an applied state, the strap descends from the front end of the shoulder portion to an elbow proximal part of the forearm on an ulnar side, the first end of the strap is fixed to the elbow pad, and the second end of the strap is fixed to the elbow pad from the back end of the shoulder portion through an axilla, traverses a front side of an upper arm, and connects to a radial side of the forearm.

10. The therapeutic shoulder brace according to claim 7, wherein the second end of the strap is fixed to the elbow pad at a position closer to a front side than a longitudinal midpoint (M) of the elbow pad.

11. The therapeutic shoulder brace according to claim 8, wherein the second end of the strap is fixed to the elbow pad at a position closer to a front side than a longitudinal midpoint (M) of the elbow pad 30.

12. The therapeutic shoulder brace according to claim 9, wherein the second end of the strap is fixed to the elbow pad at a position closer to a front side than a longitudinal midpoint (M) of the elbow pad.
